(19) United States
(12) Patent Application Publication
(10) Pub. No.: US 2018/0140169 A1
(43) Pub. Date: May 24, 2018

(54) ATTACHMENT FOR ENDOSCOPE, ENDOSCOPE SYSTEM AND METHOD FOR INDWELLING INDWELLING TOOL IN LUMINAL TISSUE

(71) Applicant: OLYMPUS CORPORATION, Tokyo (JP)

(72) Inventor: Takayasu MIKKAICHI, Tokyo (JP)

(73) Assignee: OLYMPUS CORPORATION, Tokyo (JP)

(21) Appl. No.: 15/874,620

(22) Filed: Jan. 18, 2018

Related U.S. Application Data
(63) Continuation of application No. PCT/JP2015/071210, filed on Jul. 27, 2015.

(51) Int. Cl. A61B 1/00; A61B 17/34

(52) U.S. Cl. CPC A61B 1/00101; A61B 2017/00296; A61B 17/3478; A61B 1/00094

(57) ABSTRACT

An attachment for an endoscope includes: a tubular main body having a proximal end portion; a partition part provided in the main body, the partition part being provided at a position at which a distance from the proximal. end portion to the partition part is longer than a protrusion length of a distal end portion of a device in contact with the partition part toward a proximal end side of the main body, to prevent the distal end portion of the device entering from a distal end side of the main body from coming into contact with a distal end surface of the insertion part; and a communication part provided in the partition part, the communication part forming a communication passage through which a proximal end side and a distal end side of the partition part communicate with each other in the main body.

This application is a continuation application based on PCT Patent Application No. PCT/JP2015/071210, filed Jul. 27, 2015, the content of which is incorporated herein by reference.

### BACKGROUND OF THE INVENTION

### Field of the Invention

The present invention relates to an attachment for an endoscope attached to an endoscope, an endoscope system, and a method for indwelling an indwelling tool in a luminal tissue.

### Description of the Related Art

In the related art, in a treatment of an early malignant tumor or the like, a procedure for endoscopically resecting a lesion occurring on a mucous membrane in a luminal organ such as a digestive tract is performed like, for example, an endoscopic mucosal resection (EMR), an endoscopic submucosal dissection (ESD), or the like. When the lesion has grown under a submucosal layer, extensive resection is surgically performed in consideration of the risk of metastasis of the tumor in many cases. Although the surgical resection is highly invasive and the quality of life (QOL) of a patient is greatly impaired, the surgical resection may also actually be performed even for cases in which there is no concern of metastasis.

In recent years, local resection of a lesion has begun to be considered from a viewpoint that optimal treatment should be performed for a patient. When a lesion is locally resected in a laparotomy or a laparoscopic surgery, since it is difficult to identify the position of the lesion from an abdominal cavity side, resecting needs to be performed by taking a large margin with respect to a lesion itself.

Thus, the position of the lesion is confirmed from an inside of a luminal organ using an endoscope, and a certain area of the luminal organ including a margin is indicated from the inside of the luminal organ so that a procedure of resecting a necessary minimum area from the abdominal cavity side, that is, an outside of the luminal organ has been attempted.

Several conventional techniques for indicating a specific position from an inside of a luminal organ such that it is visually recognizable from an abdominal cavity side are known. For example, there is an India ink injection in which India ink is locally injected into a submucosal layer at a specific position, a method of prodding a specific position using a distal end of an endoscopic treatment instrument such as a forceps or a high-frequency knife and causing the specific position to protrude toward an abdominal cavity side, or the like. In addition, there is also a method (for example, refer to Japanese Unexamined Patent Application, First Publication No. 2005-218680) of applying light of which a luminous flux is focused from an inside of a luminal organ to a specific position and observing the specific position from an abdominal cavity side or a method of indwelling a marker such as a metallic coil or tag at a specific position so as to penetrate a luminal organ.

It is necessary to pay attention to “layer shift” in a wall of the luminal organ when these marking procedures are performed. For example, in a tissue of a stomach wall, a mucosal part from the innermost surface to a submucosal layer and a muscular layer part from a muscular layer to a serous membrane are loosely connected via connective tissue or the like of a boundary part. For this reason, the mucosal part and the muscular layer part move relative to each other in a plane direction orthogonal to a thickness direction of the stomach wall and thus layer shift easily occurs.

### SOLUTION TO PROBLEM

An attachment for an endoscope according to a first aspect of the present invention includes: a tubular main body having a proximal end portion which is attachable to an insertion part of the endoscope; a partition part provided in the main body, the partition part being provided at a position at which a distance from the proximal end portion to the partition part is longer than a protrusion length of a distal end portion of a device in contact with the partition part toward a proximal end side of the main body in a direction in which a central axis of the main body extends, to prevent the distal end portion of the device entering from a distal end side of the main body from coming into contact with a distal end surface of the insertion part; and a communication part provided in the partition part, the communication part forming a communication passage through which a proximal end side and a distal end side of the partition part communicate with each other in the main body together with an inner wall of the main body from the partition part to a distal end of the main body, in a state in which the partition part is in close contact with a biological tissue by suctioning the biological tissue.

In a second aspect of the present invention, in the attachment for the endoscope according to the first aspect, the partition part play be continuously connected to the inner wall of the main body and extend in a direction which is orthogonal to the central axis of the main body.

In a third aspect of the present invention, the attachment for the endoscope according to the first aspect, the communication part may be a through-hole located on the central axis of the main body.

In a fourth aspect of the present invention, in the attachment for the endoscope according to the first aspect, the partition part may be constituted of a plurality of linear members connected to the inner wall of the main body.

In a fifth aspect of the present invention, in the attachment for the endoscope according to the third aspect, the partition part may have a plurality of holes formed around the through-hole.

In a sixth aspect of the present invention, in the attachment for the endoscope according to the first aspect, the partition part may be provided at a position at which a distance from a distal end surface of the main body to the partition part is shorter than a distance from the proximal end portion of the main body to the partition part.

In a seventh aspect of the present invention, in the attachment for the endoscope according to the first aspect, the partition part may include a ring part having a diameter smaller than an inner diameter of the main body and a plurality of linear members configured to connect the ring part and the inner wall of the main body, and the ring part may have an opening located on the central axis extending from a proximal end of the main body toward the distal end of the main body, be disposed such that a distal end surface thereof is orthogonal to the central axis of the main body, and be supported by the plurality of linear members.

An endoscope system according to an eighth aspect of the present invention includes: the attachment for the endoscope according to any one of the above-described aspects; the endoscope including a suction part in which a suction port is provided in a distal end portion of the insertion part; and the device including a puncture part configured to puncture the biological tissue, wherein the attachment for the endoscope is attached to the distal end portion of the insertion part.

A method for indwelling an indwelling tool in a luminal tissue according to a ninth aspect of the present invention includes: by using an endoscope in which an attachment for the endoscope including a tubular main body and a partition part provided in the main body closer to a proximal end portion side than to a distal end surface of the main body and having a communication part forming a communication passage through which a proximal end side and a distal end side of the main body communicate with each other is attached to a distal end of an insertion part, a puncture device including a needle tube into which an indwelling tool is inserted, and a suction part connected to a fluid flow passage provided in the insertion part of the endoscope: inserting the insertion part of the endoscope into a patient's body; bringing a luminal tissue into close contact with the partition part by depressurizing an inside of the attachment for the endoscope using the suction part and suctioning the luminal tissue into the main body in a state in which the distal end surface of the main body of the attachment for the endoscope is in contact with a predetermined position in an inside of the luminal tissue; inserting a sheath into which the needle tube of the puncture device is inserted into a concave part formed by bringing the luminal tissue into close contact with the partition part in an outside of a lumen and bringing a distal end of the sheath into contact with a bottom surface of the concave part; puncturing the luminal tissue with the needle tube from the outside of the lumen by protruding the needle tube from the distal end of the sheath; and indwelling the indwelling tool inserted into the needle tube so as to pass through the luminal tissue.

### BRIEF DESCRIPTION OF THE DRAWINGS

FIG. 1 is a perspective view of an attachment for an endoscope according to an embodiment of the present invention.

FIG. 2 is a front view of the attachment for the endoscope according to the embodiment of the present invention.

FIG. 3 is a cross-sectional view showing a state in which the attachment for the endoscope according to the embodiment of the present invention is attached to an insertion part of an endoscope.

FIG. 4 is an overall view showing an endoscope system according to the embodiment of the present invention.

FIG. 5 is a partial cross-sectional view of a puncture device of the endoscope system according to the embodiment of the present invention.

FIG. 6 is a perspective view showing an indwelling tool according to the embodiment of the present invention.

FIG. 7 is a view showing a usage state of the endoscope system according to the embodiment of the present invention.

FIG. 8 is a view showing a usage state of the endoscope system according to the embodiment of the present invention.

FIG. 9 is a view showing a usage state of the endoscope system according to the embodiment of the present invention.

FIG. 10 is a view showing a usage state of the endoscope system according to the embodiment of the present invention.

FIG. 11 is a view showing a usage state of the endoscope system according to the embodiment of the present invention.

FIG. 12 is a view showing a usage state of the endoscope system according to the embodiment of the present invention.

FIG. 13 is a front view illustrating a first modified example of the attachment for the endoscope of FIG. 1.

FIG. 14 is a front view illustrating a second modified example of the attachment for the endoscope of FIG. 1.

FIG. 15 is a front view illustrating a third modified example of the attachment for the endoscope of FIG. 1.

FIG. 16 is a front view illustrating a fourth modified example of the attachment for the endoscope of FIG. 1.

### DETAILED DESCRIPTION OF THE PREFERRED EMBODIMENTS

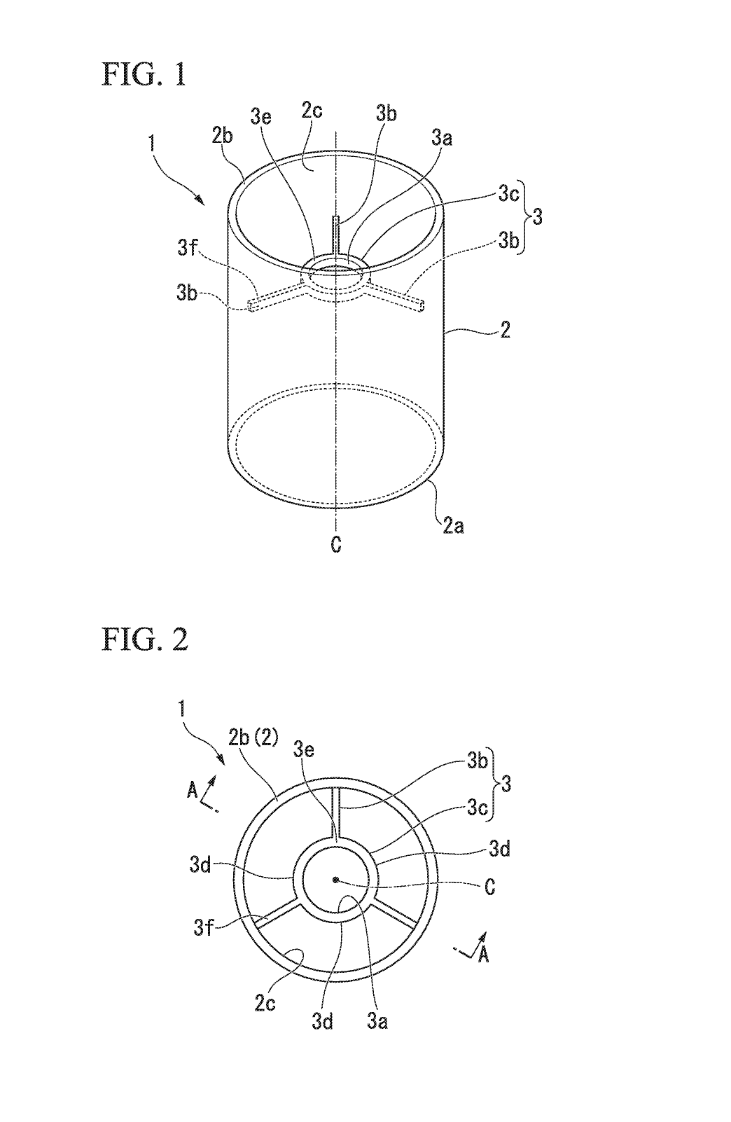

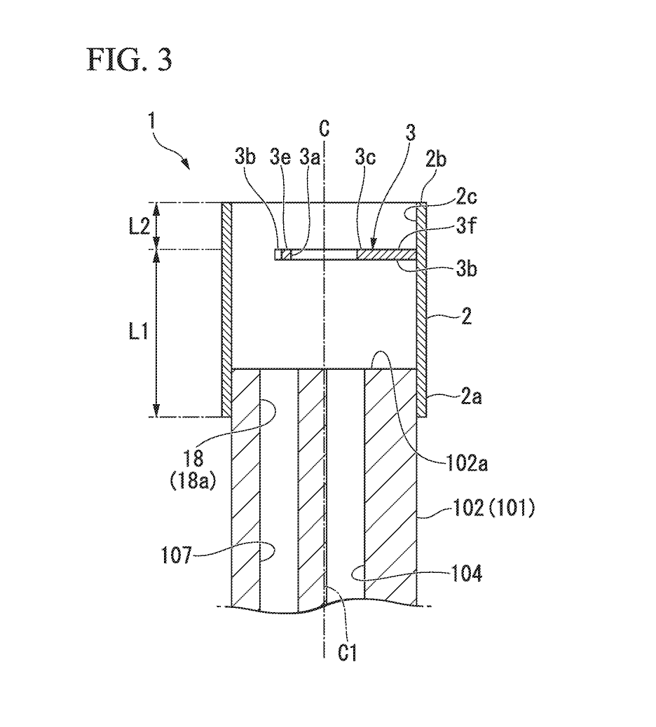

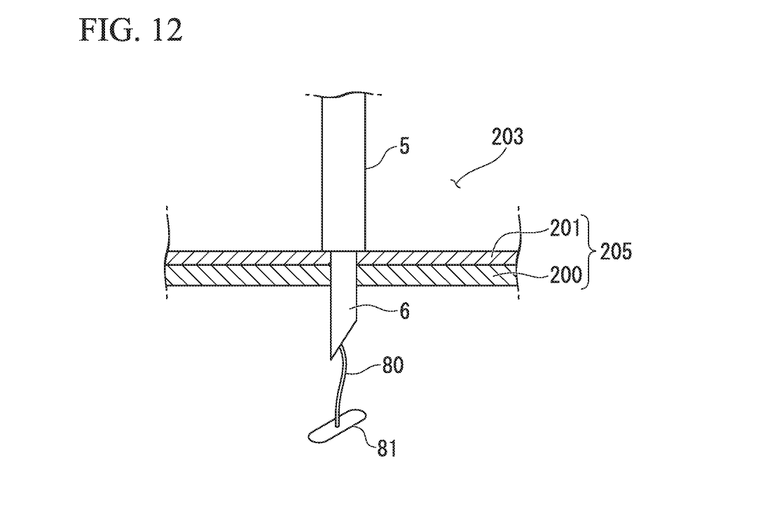

Embodiments of an attachment for an endoscope (hereinafter simply referred to as an “attachment”) and an endoscope system according to the present invention will be described below with reference to FIGS. 1 to 12. FIG. 1 is a perspective view of an attachment 1 according to an embodiment, and FIG. 2 is a front view of the attachment 1 viewed from a direction of a central axis C of the attachment 1. FIG. 3 is a view showing a state in which the attachment 1 is attached to an insertion part 102 of an endoscope 101 and is a cross-sectional view at a position indicated by line A-A of FIG. 2. Hereinafter, in the endoscope 101 and the attachment 1 described below, the insertion part 102 side with respect to an operation part 103 of the endoscope 101 and the operation part 103 side of the endoscope 101 with respect to the insertion part 102 are referred to as a distal end side and a proximal end side, respectively.

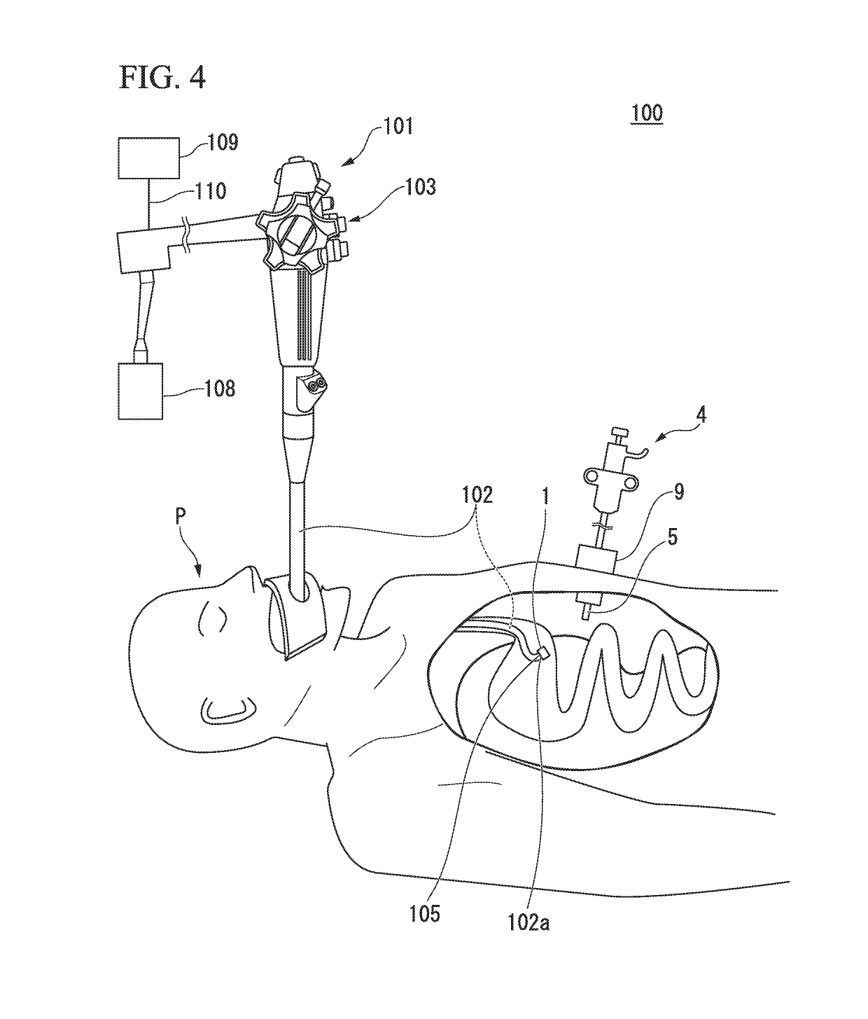

As shown in FIG. 3 and FIG. 4, the attachment 1 according to the embodiment is a member which is attachable to and detachable from a distal end of the insertion part 102 of the endoscope 101 and is attached to the distal end of the insertion part 102 and used. As shown in FIG. 1, the attachment 1 includes a tubular main body 2 and a partition part 3. The main body 2 has a proximal end portion 2a attachable to the insertion part 102 of the endoscope 101. As shown in FIGS. 1 and 2, the partition part 3 is provided in the main body 2 closer to the proximal end portion 2a side than to a distal end surface 2b of the main body 2.

As shown in FIGS. 2 and 3, the partition part 3 includes a ring part 3c and three linear members (linear parts) 3b configured to connect the ring part 3c and an inner wall 2c. The ring part 3c has a diameter smaller than an inner diameter of the main body 2. The ring part 3c is disposed such that an opening is located on the central axis C extending from the proximal end portion 2a of the main body 2 toward a distal end portion thereof and a distal end surface 3e is disposed to be orthogonal to the central axis C and is supported by three linear members 3b. The three linear members 3b are disposed at equal intervals in a circumferential direction of the ring part 3c. A through-hole (a communication part) 3a of the ring part 3c is located on the central axis C. The through-hole 3a is provided such that a proximal end side and a distal end side of the main body 2 communicate with each other so that a fluid is capable of moving from the proximal end side to the distal end side. Furthermore, in addition to the through-hole 3a of the ring part 3c, the proximal end side and the distal end side of the main body 2 also communicate with each other between the three linear members 3b, and three holes 3d are armed around the through-hole 3a of the ring part 3e.

The partition part 3 is continuously connected to the inner wall 2c of the main body 2 and extends in a direction which is orthogonal to the central axis C of the main body 2. In other words, the three linear members 3b connected to the ring part 3c are connected to the inner wall 2c, and the distal end surface 3e of the ring part 3c and a distal end surface 3f of the three linear members 3b are formed to be flush with each other in the direction which is orthogonal to the central axis C.

The main body 2 and the partition part 3 are formed of transparent embers made of a resin or the like to secure a field of view for an imaging unit described below. The partition part 3 is integrally formed with the main body 2.

As shown in FIG. 3, the partition part 3 is provided at a position at which a distance L2. from the distal end surface 2b of the main body 2 to the partition part 3 is shorter than a distance L1 from the proximal end portion 2a to the partition part 3 in the direction of the central axis C. Furthermore, the distance L2 is set to be longer than a protrusion length of a needle tube of a puncture device described below.

An endoscope system 100 using the attachment 1 will be described below.

As shown in FIG. 4, the endoscope system 100 according to the embodiment includes the attachment 1, the endoscope 101, and a puncture device 4. A well-known endoscope is used as the endoscope 101. As shown in FIG. 3, when the attachment 1 is attached to a distal end portion of the insertion part 102 of the endoscope 101, the main body 2 is configured so that the central axis C is coaxial with a longitudinal axis C1 of the insertion part 102, protrudes from the distal end portion of the insertion part 102, and extends along the longitudinal axis C1.

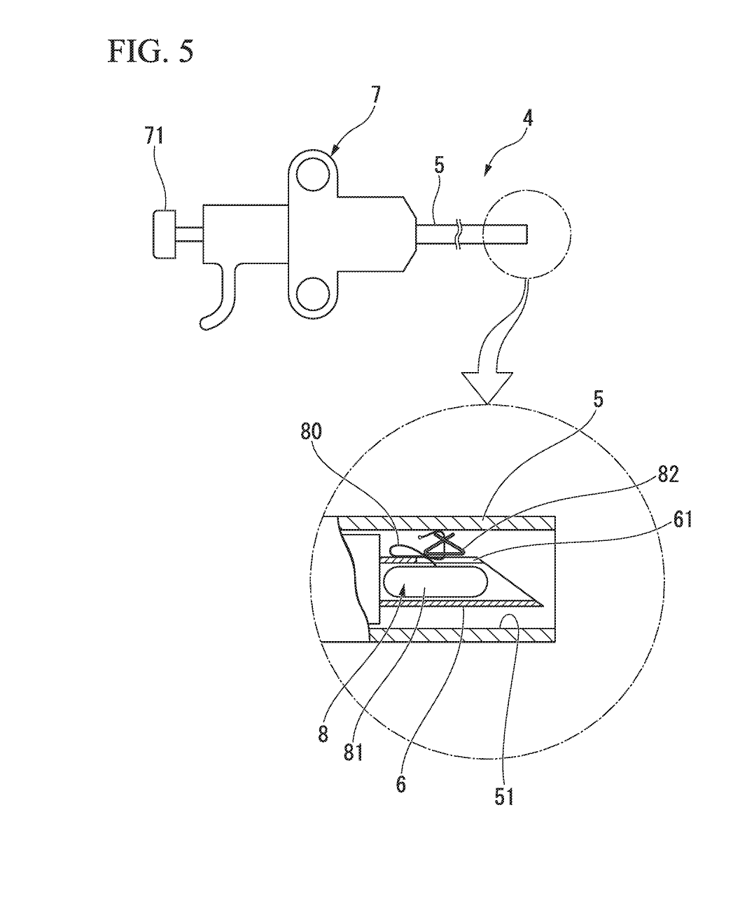

FIG. 5 illustrates a side view including a partial cross-sectional view of the puncture device 4. In the puncture device 4, an outer sheath 5 side with respect to an operation part 7 and the operation part 7 side with respect to the outer sheath 5 are referred to as a distal end side and a proximal end side, respectively.

In the puncture device 4, a needle tube 6 (a puncture part) is inserted into the elongated outer sheath 5 extending from the operation part 7. In the needle tube 6, a pusher (not shown) is disposed in the needle tube 6 and is capable of moving forward and backward, and a well-known indwelling tool 8 is disposed on the distal end side of the pusher.

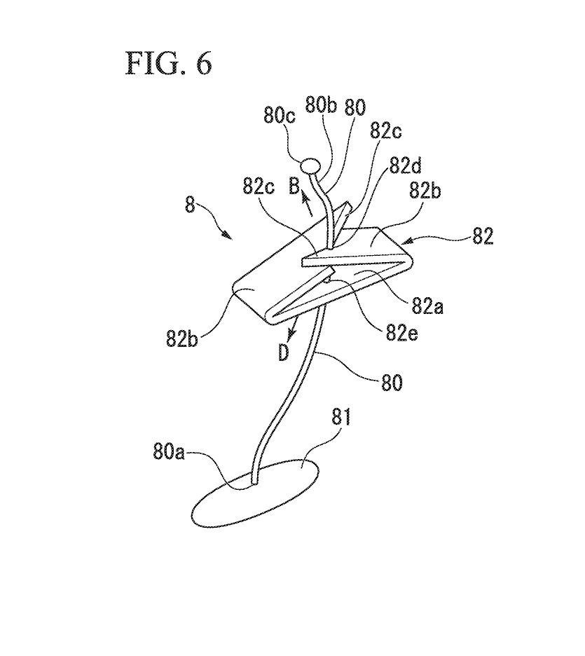

FIG. 6 is a perspective view of the indwelling tool 8. As shown in FIG. 6, the indwelling tool 8 includes a thread 80 inserted through a biological tissue, an anchor 81, and a locking member 82. The anchor 81 is constituted of a cylindrical member, and a first end 80a of the thread 80 is connected to the anchor 81 at the vicinity of the center in a longitudinal direction of the anchor 81.

The locking member 82 includes a base part 82a located at the center thereof and two bent pieces 82b located at both ends of the base part 82a in a longitudinal direction thereof. The locking member 82 is formed by bending both end portions of a substantially parallelogram-shaped plate-like member in a longitudinal direction thereof An opening 82e is formed in the center of the base part 82a and the thread 80 is inserted through the opening 82e. The pair of bent pieces 82b are bent at a predetermined angle which is an acute angle with respect to the base part 82a, and inclined edge portions 82c face and are in proximity to each other. Cutouts 82d are formed in the edge portions 82c of the pair of bent pieces 82b. The cutouts 82d face each other in a state in which the edge portions 82c of the pair of bent pieces 82b are close to each other, and a second end 80b side of the thread 80 is inserted into a clearance between the cutouts 82d. A knot used as a stopper 80c is formed at the second end 80b of the thread 80. The stopper 80c may be formed by enlarging an end of the thread 80 in a spherical shape by melting the thread 80.

When the second end 80b of the thread 80 is pulled in a direction (a direction of an arrow B shown in FIG. 6) which is away from the anchor 81 it a state in which the locking member 82 is fixed, the pair of bent pieces 82b are elastically deformed in directions in which the bent angles thereof increase so that the clearance between the cutouts 82d is enlarged and thus the thread 80 is moved toward the distal end side. On the other hand, when the thread 80 is pulled toward the first end 80a side (in a direction of an arrow D shown in FIG. 6), the pair of bent pieces 82b are elastically deformed in directions in which the bent angles thereof decrease so that the clearance between the cutouts 82d is reduced and the movement of the thread 80 is restricted. In other words, when the thread 80 is pulled toward the second end 80b side, the locking member 82 is moved in a direction in which the locking member 82 approaches the anchor 81. On the other hand, since the movement of the locking member 82 in a direction in which the locking member 82 is moved away from the anchor 81 is restricted, a state in which the locking member 82 and the anchor 81 are close to each other is maintained. The locking member 82 functions as a stopper of the thread 80 after the thread 80 is inserted through the biological tissue.

In the partial cross-sectional view shown in FIG. 5, in the indwelling tool 8, the anchor 81 is inserted into the needle tube 6 and is disposed in a lumen 51 of the outer sheath 5 in a state in which the thread 80 and the locking member 82 are disposed in an outer circumferential edge of the needle tube 6 from a slit 61 formed in a side surface of the needle tube 6 to extend in a long axis direction.

A configuration of the endoscope 101 is not particularly limited. For example, in the embodiment, the endoscope 101 includes the flexible insertion part 102 and the operation part 103 for the endoscope 101 attached to a proximal end portion of the insertion part 102.

The insertion part 102 is formed in a cylindrical shape. A plurality of channels 104 extending in a longitudinal direction are formed in the insertion part 102. An observation unit 105, a fluid flow passage 107, and a light guide 106 are provided in each of the plurality of channels 104.

The observation unit 105 includes an imaging element (not shown) such as a complementary metal-oxide semiconductor (CMOS) image sensor. A display 109 is connected to the operation part 103 via a universal cable 110. An image to be observed in a predetermined field of view acquired by the imaging element is converted into a signal and transmitted to the display 109.

One of the plurality of channels 104 is used as the fluid flow passage 107. The fluid flow passage 107 communicates in a longitudinal direction of the insertion part 102. An opening in a proximal end portion of the fluid flow passage 107 is connected to a suction source 108 connected to the operation part 103 of the endoscope 101. The endoscope 101 includes a suction part 18 in which a suction port 18a is provided in the distal end portion of the insertion part 102. The suction part 18 (suction port 18a) is connected to the fluid flow passage 107.

The light guide 106 is provided in a distal end surface 102a, of the insertion part 102, is inserted into the fluid flow passage 107, and extends to the proximal end side. The light guide 106 is provided to emit illumination light in front of the insertion part 102 at the time of imaging using the imaging element.

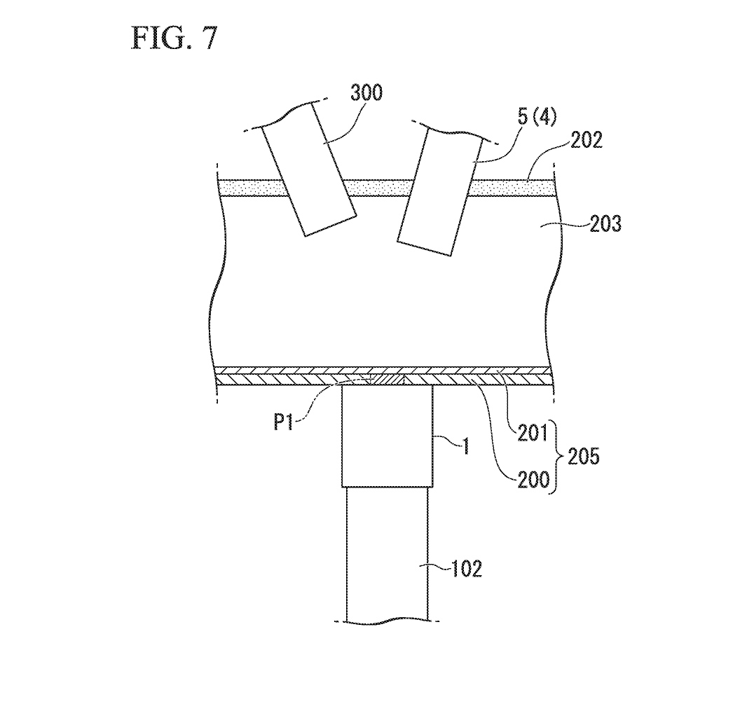

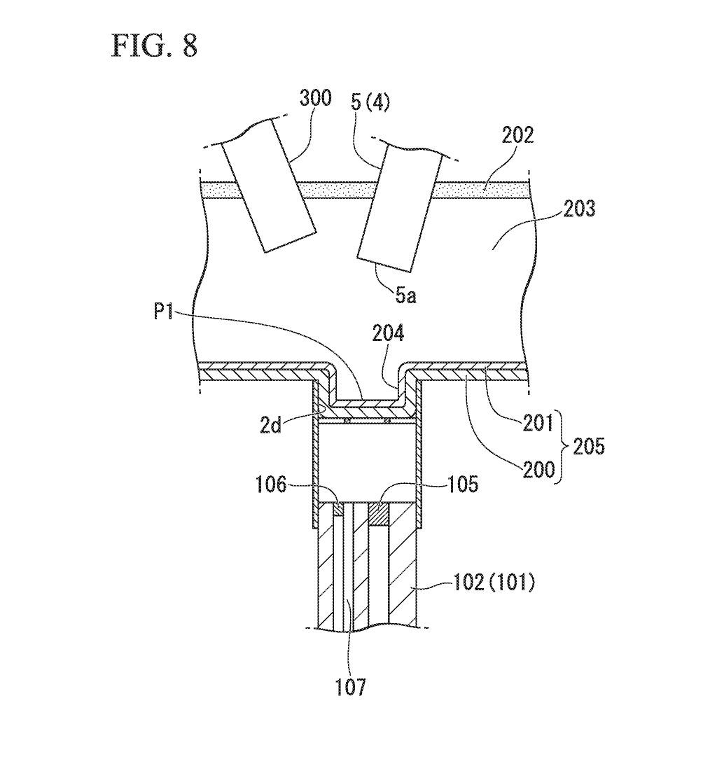

A procedure using the endoscope system 100 will be described below with reference to FIGS. 7 to 12. In such a procedure, a procedure of identifying an area to be treated and indicating a marking position P1 in the vicinity of the area from an inside of a luminal organ using the endoscope 101 to which the attachment 1 is attached, and indwelling the indwelling tool 8 using the puncture device 4 inserted from an abdominal cavity side in response to an instruction from the endoscope 101 is performed. Note that, in FIGS. 8, 10, and 11, the positions of the linear members 3b of the partition part 3 are indicated using imaginary lines. Furthermore, in FIGS. 7 to 12, for convenience of explanation, a luminal tissue 205 is simply referred to as a mucosal layer 200 and a muscular layer 201.

First, an operator orally inserts the insertion part 102 of the endoscope 101 to which the attachment 1 is attached into a patient's body. On the other hand, a laparoscope 300 and the puncture device 4 are inserted into an abdominal wall 202. The laparoscope 300 and the puncture device 4 are inserted into a trocar 9 (refer to FIG. 4) inserted and indwelled into the abdominal wall 202 and are inserted into an abdominal cavity 203, but in FIGS. 7 and 8, the configurations thereof are omitted and shown schematically. The operator brings the attachment 1 into contact with the mucosal layer 200 (a mucous membrane) at the marking position P1 on the tissue while checking an image of the observation unit 105 in the endoscope 101 on the display 109.

Subsequently, when the operator drives the suction source 108 to start suction, an inside of the main body 2 is depressurized in a state in which the mucosal layer 200 (the mucous membrane) is in contact with the distal end surface 2b of the attachment 1, and as shown in FIG. 8, the luminal tissue 205 is suctioned to a position where the luminal tissue 205 comes into contact with the partition part 3 in the main body 2 of the attachment 1. At this time, since the proximal end side and the distal end side of the main body 2 communicate with each other through the through-hole 3a of the ring part 3c and the three holes 3d around the through-hole 3a in the partition part 3, the suctioned luminal tissue 205 is brought into close contact with the distal end surface 2b of the main body 2, a surface of the partition part 3 on the distal end side, and a distal-end-side inner wall 2d between the distal end surface 2b and the partition part 3. In other words, the through-hole (communication part) 3a is capable of forming a communication passage through which the proximal end side and the distal end side of the main body 2 (the partition part 3) communicate with each other in the main body 2 together with the inner wall 2c (the distal-end-side inner wall 2d) of the main body 2 from the partition part 3 to a distal end of the main body in a state in which the partition part 3 is in close contact with the luminal tissue 205 by suctioning the luminal tissue 205. Furthermore, the luminal tissue 205 is suctioned with a strong suction force and is supported so that layer shift between the mucosal layer 200 and the muscular layer 201 is unlikely to occur.

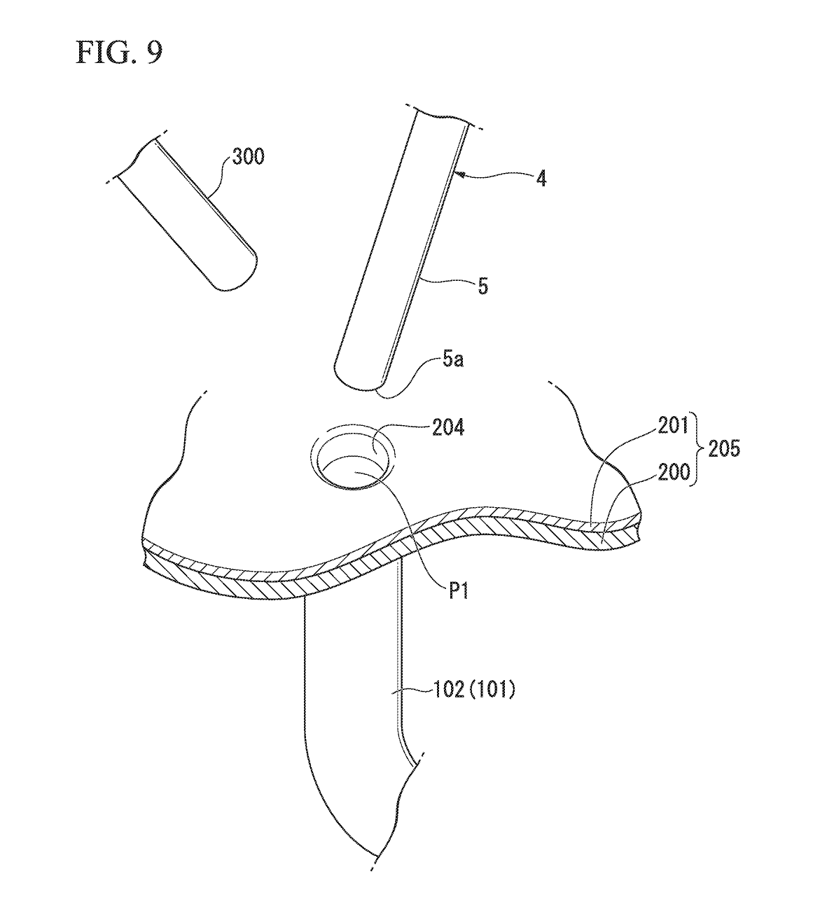

At this time, on the abdominal cavity 203 side, as shown in FIG. 9, a state in which a concave part 204 is formed so that the luminal tissue 205 is concave along a distal end shape of the attachment 1 is capable of being visually recognized by the laparoscope 300. The operator brings a distal end portion 5a of the outer sheath 5 in the puncture device 4 close to the concave part 204 of the mucosal layer 200 and the muscular layer 201 while checking an image captured by the laparoscope 300 with a monitor (not shown).

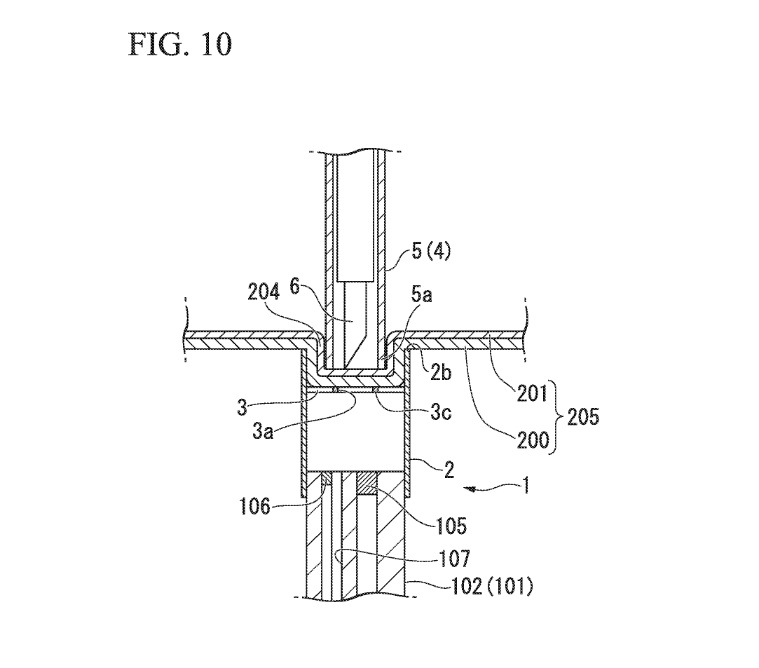

Next, as shown in FIG. 10, the distal end portion 5a of the outer sheath 5 is inserted into the concave part 204 and brought into contact with the muscular layer 201 on a bottom surface of the concave part 204. A diameter of the attachment 1 on the distal end surface 2b side is set so that the distal end portion 5a of the outer sheath 5 is capable of being inserted into the concave part 204 when the mucosal layer 200 is brought into close contact with the distal-end-side inner wall 2d of the main body 2 in consideration of a thickness of the luminal tissue 205. For this reason, since the operator is capable of inserting the distal end portion 5a of the outer sheath 5 to engage the distal end portion 5a with the concave part 204 while viewing an image of the laparoscope 300, the distal end portion 5a of the outer sheath 5 is capable of being easily disposed at an indicated position from the endoscope 101 side.

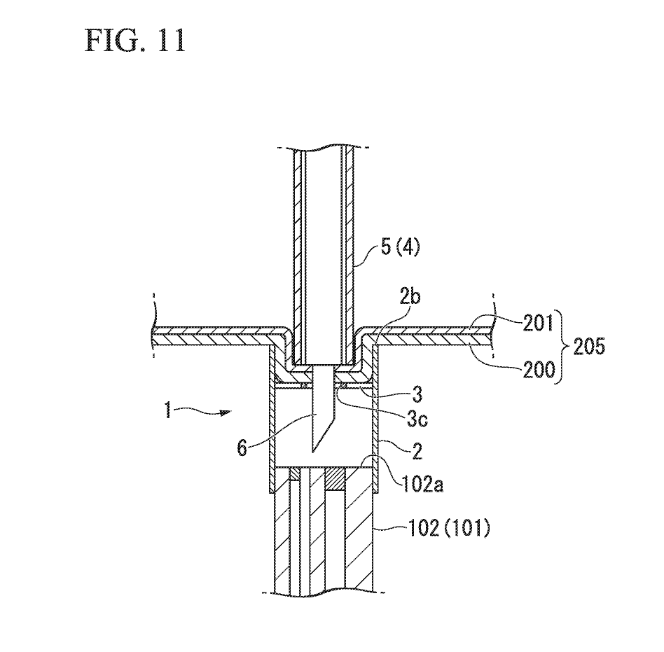

Next, as shown in FIG. 11, the operator punctures the luminal tissue 205 by causing the needle tube 6 disposed in the lumen 51 of the outer sheath 5 to protrude from a distal end of the outer sheath 5. The needle tube 6 is inserted into the through-hole 3a of the ring part 3c located on the central axis C. Furthermore, since the luminal tissue 205 and the distal end of the outer sheath 5 are supported by the partition part 3, the needle tube 6 stably punctures the luminal tissue 205. In addition, since the partition part 3 is provided at a position at which the distance L2 is shorter than the distance L1 in the direction of the central axis C, it is possible to prevent a distal end of the needle tube 6 from coming into contact with the distal end surface 102a of the insertion part 102 (FIG. 11). In other words, the partition part 3 is provided at a position at which a distance from the proximal end portion 2a to the partition part 3 is longer than a protrusion length of a distal end portion of the puncture device 4 in contact with the partition part 3 toward the proximal end side of the main body 2 in a direction in which the central axis C of the main body 2 extends, to prevent the distal end portion of the puncture device 4 entering from the distal end side of the main body 2 from coming into contact with the distal end surface 102a of the insertion part 102.

In this state, the operator pushes a pusher operation part 71 of the operation part 7 in the puncture device 4 to move forward the pusher (not shown) against the needle tube 6 and thus the anchor 81 is pushed outside of the needle tube 6. Next, when a depressurized state in the attachment 1 is released by stopping the driving of the suction source 108, the close contact between the distal end surface 2b of the attachment 1 and the luminal tissue 205 is released. The operator moves the insertion part 102 backward.

Subsequently, the indwelling tool 8 is indwelled in the luminal tissue 205 using a well-known method. In other words, the needle tube 6 is moved backward toward the abdominal cavity 203 side in a state in which the outer sheath 5 is brought into contact with the muscular layer 201, Subsequently, when the outer sheath 5 is moved backward, the locking member 82 falls off from the lumen 51 of the outer sheath 5 toward the abdominal cavity 203 side. Subsequently, an operation such as pulling the second end 80b of the thread 80 toward the abdominal wall 202 side is performed using a grasping forceps (not shown) inserted from the abdominal cavity 203 side. As a result, the thread 80 passes through the luminal tissue 205 and the indwelling tool 8 is indwelled in a state in which the luminal tissue 205 is sandwiched between the anchor 81 and the locking member 82.

Since a plurality of marking positions P1 are set around an area to be treated, a plurality of indwelling tools 8 are indwelled around the area to be treated by repeating the above-described procedure a plurality of times and thus the area to be treated is capable of being identified from the abdominal cavity 203 side. The operator manipulates the grasping forceps to endoscopically take out a lesion mucosal part and sutures the surroundings to complete a series of treatments.

As described above, according to the attachment 1 and the endoscope system 100 of the embodiment, the marking which is capable of being visually recognized from the inside and the outside (the abdominal cavity 203 side) of the luminal organ is capable of being appropriately provided to a specific position on the luminal organ by attaching the attachment 1 to an existing endoscope device.

Since the partition part 3 is provided in the main body 2 closer to the proximal end portion 2a side than to the distal end surface 2b of the main body 2, when the inside of the main body 2 is depressurized in a state in which the distal end surface 2b of the main body 2 is brought into contact with the mucosal layer 200, the concave part 204 is formed such that the luminal tissue 205 is suctioned between the distal end surface 2b and the partition part 3. For this reason, the marking position P1 is capable of being easily identified from the abdominal cavity 203 side.

Since the partition part 3 having the through-hole (the communication part) 3a is provided in the attachment 1, when the inside of the main body 2 is depressurized through suction in a state in which the distal end surface 2b of the main body 2 is in contact with the mucosal layer 200 (the mucous membrane), a state in which the luminal tissue 205 is suctioned into the main body 2 and is in close contact with the partition part 3 is maintained. As a result, the marking position P1 is capable of being easily identified on the abdominal cavity 203 side while preventing layer shift between the mucosal layer 200 and the muscular layer 201.

Since the partition part 3 is continuously connected to the inner wall 2c of the main body 2 and extends in the direction which is orthogonal to the central axis C of the main body 2, the bottom surface of the concave part 204 is formed to be orthogonal to the central axis C. The outer sheath 5 is stably supported when the distal end portion 5a of the outer sheath 5 in the puncture device 4 is inserted and in contact with the bottom surface of the concave part 204. In addition, at this time, since a central axis of the outer sheath 5 and the central axis C of the attachment 1 are coaxial with or parallel to each other, the needle tube 6 is capable of stably puncturing the luminal tissue 205 in the direction of the central axis C.

Since the through-hole 3a located on the central axis C is provided as a communication part in the partition part 3, when the outer sheath 5 of the puncture device 4 is inserted into the concave part 204 and thus the needle tube 6 punctures the luminal tissue 205, it is possible to prevent the partition part 3 from hindering the puncturing of the needle tube 6.

Since the partition part 3 includes three linear members 3b, the ring part 3c is capable of being stably supported. Furthermore, since a plurality of large holes 3d serving as fluid flow passages are formed in addition to the through-hole 3a, a suction force by the suction source 108 is hardly disturbed. Therefore, the luminal tissue 205 is capable of being brought into close contact with the partition part 3 at the time of suction.

Also, the operator is capable of easily aligning the position of the needle tube 6 at the marking position P1 by inserting the distal end portion 5a of the outer sheath 5 in the puncture device 4 from the abdominal cavity 203 side into the concave part 204.

Since the needle tube 6 punctures the luminal tissue 205 while maintaining a suction state, it is possible to prevent the occurrence of layer shift between the mucosal layer 200 and the muscular layer 201 when the needle tube 6 punctures the luminal tissue 205.

According to the endoscope system 100, the marking position P1 identified in the lumen is capable of being easily visually recognized from the abdominal cavity side. In addition, the indwelling tool 8 is capable of being indwelled while preventing layer shift between the mucosal layer 200 and the muscular layer 201.

The configuration of the attachment 1 according to the embodiment is capable of being variously modified as described below.

Although an example in which the partition part 3 is constituted of the ring part 3c and the three linear members 3b has been described, the configuration of the partition part is not limited thereto. For example, the number of linear members 3b may be two or four or more. Here, in order to stably form a stereotactic plane in the suctioned luminal tissue 205 at the time of suction, it is preferable that the partition part 3 is constituted of at least three or more linear members. Furthermore, in the partition part 3, the linear members 3b and the ring part 3c may be formed of wires and fixed to the inner wall 2c. In addition to the configuration in which the partition part 3 is integrally formed with the main body 2, a configuration in which the linear members 3b are engaged with or joined to the inner wall 2c of the main body 2 may be adopted.

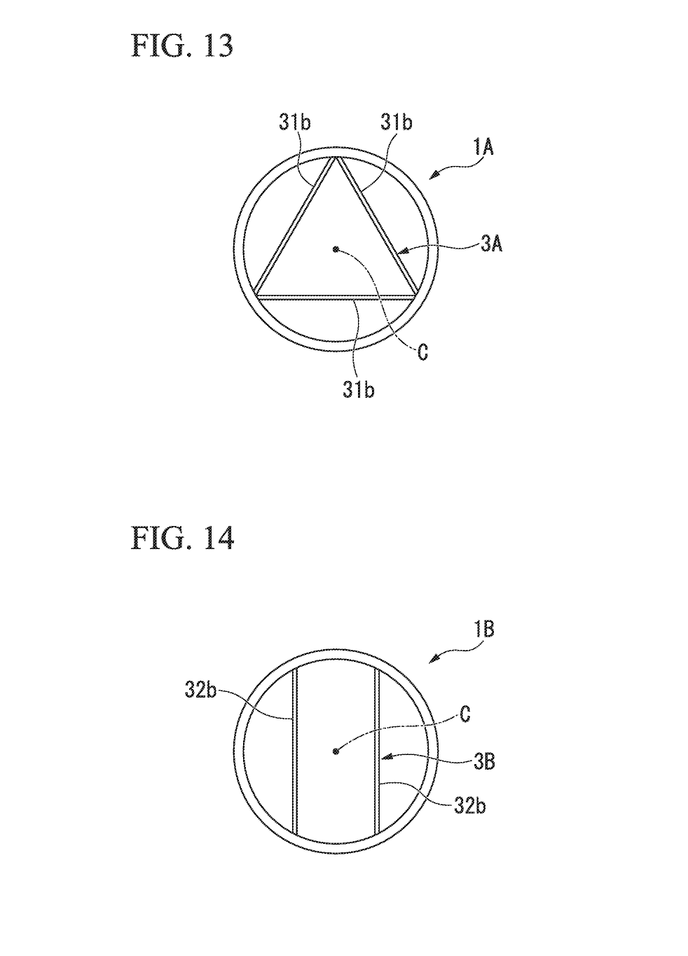

FIGS. 13 to 16 illustrate front views of first to fourth modified examples of the attachment 1 according to the embodiment. Attachments 1A to 1D in the first to fourth modified examples are different from shapes of the partition part 3. The configuration is otherwise the same as that in the above-described embodiment.

For example, like the attachment 1A in the first modified example illustrated in FIG. 13, a triangular partition part 3A surrounding the central axis C may be formed by three linear members 31b. In this case, an inside of the triangular shape serves as a communication part and three holes are formed around the communication part. In such a partition part 3A as well, the suctioned luminal tissue 205 is brought into close contact with the partition part 3A to form the concave part 204 having the bottom surface.

For example, like in the attachment 1B of the second modified example illustrated in FIG. 14, a partition part 3B may be formed by two linear members 32b disposed in parallel with the central axis C sandwiched therebetween. In such a partition part 3B as well, the suctioned luminal tissue 205 is brought into close contact with the partition part 3B to form the concave part 204 having the bottom surface.

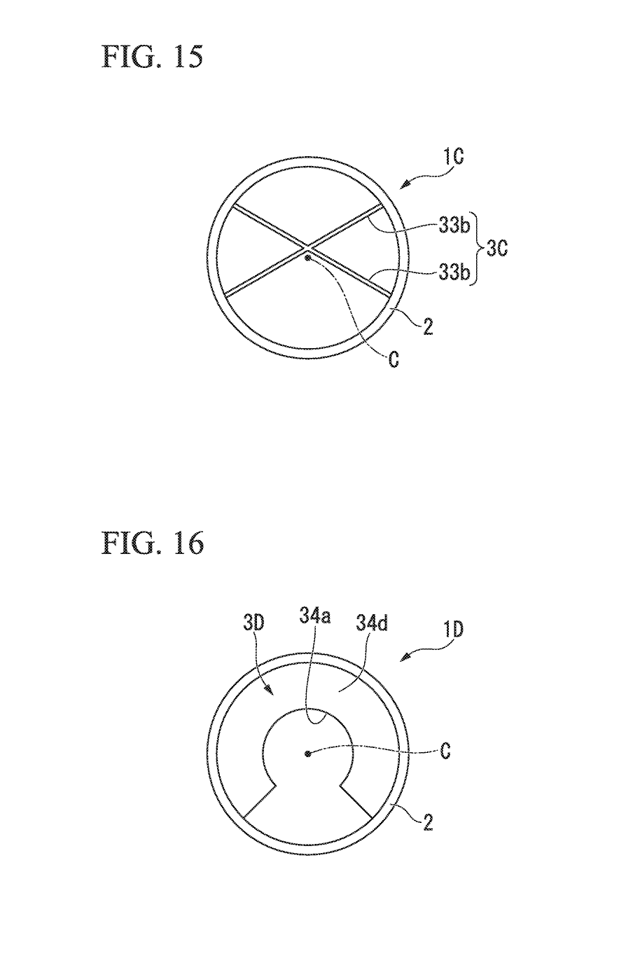

For example, like in the attachment 1C of the third modified example illustrated in FIG. 15, a partition part 3C may be formed by two linear members 33b intersecting each other avoiding the central axis C. In this case, a point of intersection of the two linear members 33b is at a position shifted from the central axis C, the communication part is located on the central axis C, and four linear members are formed with the point of intersection of the linear members 33b as a boundary. In such a partition part 3C as well, the suctioned luminal tissue 205 is brought into close contact with the partition part 3C to form the concave part 204 having the bottom surface.

For example, like in the attachment ID of the fourth modified example illustrated in FIG. 16, the partition part 3D having a surface portion 34d formed by cutting a part of a ring-shaped flat plate in a circumferential direction thereof may be formed. In the partition part 3D, a circular-arc-shaped opening 34a is formed around the central axis C and the opening 34a and a cut portion of the surface portion 34d in a circumferential direction thereof serves as a communication part. In this case, the opening 34a is formed to have a size in which the outer sheath 5 of the puncture device 4 inserted into the concave part 204 from the abdominal cavity 203 side is capable of being supported.

In addition, the indwelling tool 8 illustrated in the embodiment may be indwelled in a tissue to function as a marker. For example, an elastic wire that is capable of being restored to a coil shape may be pierced into the tissue via the needle tube 6.

While the embodiments of the present invention have been described above, the technical scope of the present invention is not limited to the above-described embodiments, and it is possible to change combinations of the constituent elements in the embodiments, variously change each constituent element, or delete each constituent element without departing from the gist of the present invention. The present invention is not limited by the foregoing description.

What is claimed is:

1. An attachment for an endoscope comprising:
a tubular main body having a proximal end portion which is attachable to an insertion part of the endoscope;
a partition part provided in the main body, the partition part being provided at a position at which a distance from the proximal end portion to the partition part is longer than a protrusion length of a distal end portion of a device in contact with the partition part toward a proximal end side of the main body in a direction in which a central axis of the main body extends, to prevent the distal end portion of the device entering from a distal end side of the main body from coming into contact with a distal end surface of the insertion part; and
a communication part provided in the partition part, the communication part forming a communication passage through which a proximal end side and a distal end side of the partition part communicate with each other in the main body together with an inner wall of the main body from the partition part to a distal end of the main body, in a state in which the partition part is in close contact with a biological tissue by suctioning the biological tissue.

2. The attachment for the endoscope according to claim 1, Wherein the partition part is continuously connected to the inner wall of the main body and extends in a direction which is orthogonal to the central axis of the main body.

3. The attachment for the endoscope according to claim 1, wherein the communication part is a through-hole located on the central axis of the main body.

4. The attachment for the endoscope according to claim 1, wherein the partition part is constituted of a plurality of linear members connected to the inner wall of the main body.

5. The attachment for the endoscope according to claim 3, wherein the partition part has a plurality of holes formed around the through-hole.

6. The attachment for the endoscope according to claim 1, wherein the partition part is provided at a position at which a distance from a distal end surface of the main body to the partition part is shorter than a distance from the proximal end portion of the main body to the partition part.

7. The attachment for the endoscope according to claim 1, wherein the partition part includes a ring part having a diameter smaller than an inner diameter of the main body and a plurality of linear members configured to connect the ring part and the inner wall of the main body, and
the ring part has an opening located on the central axis extending from a proximal end of the main body toward the distal end of the main body, is disposed such that a distal end surface thereof is orthogonal to the central axis of the main body, and is supported by the plurality of linear members.

8. An endoscope system comprising:
the attachment for the endoscope according to claim 1;
the endoscope including a suction part in which a suction port is provided in a distal end portion of the insertion part; and
the device including a puncture part configured to puncture the biological tissue, wherein the attachment for the endoscope is attached to the distal end portion of the insertion part.

9. A method for indwelling an indwelling tool in a luminal tissue comprising:
by using an endoscope in which an attachment for the endoscope including a tubular main body and a partition part provided in the main body closer to a proximal end portion side than to a distal end surface of the main body and having a communication part forming a communication passage through which a proximal end side and a distal end side of the main body communicate with each other is attached to a distal end of an insertion part, a puncture device including a needle tube into which an indwelling tool is inserted, and a suction part connected to a fluid flow passage provided in the insertion part of the endoscope:
inserting be insertion part of the endoscope into a patient's body;
bringing a luminal tissue into close contact with the partition part by depressurizing an inside of the attachment for the endoscope using the suction part and suctioning the luminal tissue into the main body in a state in which the distal end surface of the main body of the attachment for the endoscope is in contact with a predetermined position in an inside of the luminal tissue;
inserting a sheath into which the needle tube of the puncture device is inserted into a concave part formed by bringing the luminal tissue into close contact with the partition part in an outside of a lumen and bringing a distal end of the sheath into contact with a bottom surface of the concave part;
puncturing the luminal tissue with the needle tube from the outside of the lumen by protruding the needle tube from the distal end of the sheath; and
indwelling the indwelling tool inserted into the needle tube so as to pass through the luminal tissue.
